(19) United States
(12) Patent Application Publication
(10) Pub. No.: US 2024/0093194 A1
(43) Pub. Date: Mar. 21, 2024

(54) MIR-7-5P MIMIC FOR INHIBITING MIGRATION AND INVASION OF BREAST CANCER, SCREENING METHOD AND APPLICATION THEREOF

(71) Applicant: QUFU NORMAL UNIVERSITY, Shangdong (CN)

(72) Inventors: Ge Yang, Shangdong (CN); Zhaoyi Liang, Shangdong (CN); Chengchuan Che, Shangdong (CN); Jinfeng Liu, Shangdong (CN); Zhijin Gong, Shangdong (CN)

(73) Assignee: QUFU NORMAL UNIVERSITY, Shangdong (CN)

(21) Appl. No.: 18/467,675

(22) Filed: Sep. 14, 2023

(30) Foreign Application Priority Data
Sep. 16, 2022 (CN) 202211125861.3

(51) Int. Cl. C12N 15/113; A61P 35/04; C12Q 1/6886

(52) U.S. Cl. CPC C12N 15/113; A61P 35/04; C12Q 1/6886; C12N 2310/141; C12N 2320/30; C12Q 2600/136; C12Q 2600/158; C12Q 2600/178

(57) ABSTRACT

The present invention belongs to the technical field of biomedicine, and mainly relates to a miR-7-5p mimic for inhibiting migration and invasion of breast cancer, a screening method and an application thereof. The sequence of the miR-7-5p is shown in SEQ ID NO.1. The present invention has found that the miR-7-5p mimic generates a significant inhibitory effect on breast cancer through targeted inhibition of the molecular mechanism of RYK. In vitro culture system, the miR-7-5p mimic can function to inhibit migration and invasion capabilities of breast cancer. The miR-7-5p mimic can inhibit RYK protein and mRNA levels in breast cancer. In nude mice, the miR-7-5p mimic can also significantly inhibit migration and invasion capabilities of breast cancer. Therefore, the present invention demonstrates that the miR-7-5p mimic can be a small-nucleic-acid drug to significantly inhibit breast cancer metastasis.

### CROSS-REFERENCE TO RELATED APPLICATION

This application claims the priority benefit of China application serial no. 202211125861.3, filed on Sep. 16, 2022. The entirety of the above-mentioned patent application is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on May 19, 2023, is named 132365US-sequence_listing and is 25,069 bytes in size.

### BACKGROUND

### Technical Field

The present invention belongs to the technical field of biomedicine, and mainly relates to a miR-7-5p mimic for inhibiting migration and invasion of breast cancer, a screening method and an application thereof.

### Description of Related Art

In recent years, the incidence of breast cancer has increased year by year, and breast cancer has become the biggest killer that threatens women's health. Breast cancer often results in function decline of other organs and even failure of organs which may lead to death, due to uncontrollable spread of tumor cells. Although good progress has been made in the treatment of primary breast cancer, there is no effective treatment for metastatic breast cancer. Treatments including surgery, chemotherapy, and radiotherapy are mainly suitable for patients with un-metastasized breast cancer in the early stage, and show little benefit to patients with extensive metastasis in the late stage. In addition, chemotherapy and other treatments have significant toxic and side effects.

However, recent studies have shown that exosomes can be loaded with a large number of bioactive molecules released by cells (small non-coding RNAs, mRNAs, DNAs, proteins, etc.) and enter the body fluid, which releases their contents into target cells with the flow of body fluids and their binding to membranes of the target cells, thereby playing a role in information transmission between cells. Exosome miRNAs also reflect to some extent the expression mode of aberrant miRNAs in tumor cells. Aberrant miRNAs may better show specific effects on the tumor cells. In addition, since miRNAs are small RNAs naturally occurring in organisms with the capability of targeted inhibition of gene expression via the principle of base complementarity, miRNAs have the advantage of specificity and become a powerful tool for developing targeting drugs. Therefore, screening for differentially expressing miRNAs associated with breast cancer metastasis using exosomes of differently invasive breast cancer cells allows for the development of a small-nucleic-acid drug capable of inhibiting breast cancer metastasis.

### SUMMARY

In response to the problems associated with existing treatments for metastatic breast cancer, the present invention provides a miR-7-5p mimic for inhibiting migration and invasion of breast cancer, which is capable of targeted inhibition of RYK gene expression, thereby inhibiting migration and invasion of breast cancer.

The present invention also provides a screening method for a miR-7-5p mimic for inhibiting migration and invasion of breast cancer.

It is another object of the present invention to provide application of a miR-7-5p mimic in preparation of a small-nucleic-acid drug for inhibiting migration and invasion of breast cancer.

In order to achieve the above object, the present invention employs the following technical solution.

The present invention provides a miR-7-5p mimic for inhibiting migration and invasion of breast cancer, wherein the sequence of the miR-7-5p is shown in SEQ ID NO.1: UGGAAGACUAGUGAUUUUGUUGUU.

Further, the sequence of the miR-7-5p mimic is as follows:

| | Guide strand: |
|---|---|
| (SEQ ID NO. 1) | |
| | UGGAAGACUAGUGAUUUUGUUGUU; |
| | Passenger strand: |
| (SEQ ID NO. 27) | |
| | CAACAAAAUCACUAGUCUUCCAUU. |

The present invention also provides a screening method for a miR-7-5p mimic, including the following steps:
step (1), utilizing two subsets, MDA231 EXO and MCF7 EXO, in dataset GSE114329 under GEO database for bioinformatics analysis, downloading original data from the dataset and filtering and processing the original data, and then screening for differentially expressing genes; and
step (2), subjecting the screened differentially expressing miRNAs to qRT-PCR analysis to screen out the miR-7-5p.

Further, in the step (2), the procedure of the qRT-PCR includes: pre-denaturation at 95° C. for 5 min, and 40 cycles including 95° C. for 15 sec, 60° C. for 20 sec, and 72° C. for 40 sec, with an internal reference being U6.

Further, in the qRT-PCR analysis, primer sequences of the miRNAs are specifically as follows:

| Genes | Primer sequences (5′-3′) |
|---|---|
| RYK | Forward: CTTTATCAGTGTTTCGGGTAG (SEQ ID |
| | NO. 2) |
| | Reverse: GCGTAGAAGTGGTTGGAGC (SEQ ID |
| | NO. 3) |
| GADPH | Forward: AGCCACATCGCTCAGACAC (SEQ ID |
| | NO. 4) |
| | Reverse: GCCCAATACGACCAAATCC (SEQ ID |
| | NO. 5) |
| U6 | Reverse transcription: |
| | TGGTGTCGTGGAGTCG (SEQ ID NO. 6) |
| | Forward: CTCGCTTCGGCAGCACA (SEQ ID |
| | NO. 7) |
| | Reverse: AACGCTTCACGAATTTGCGT (SEQ ID |
| | NO. 8) |
| Has-miR- | Reverse transcription: |
| 7-5p | GTCGTATCCAGTGCAGGGTCCGAGGTATTCGCACTGGAT |
| | ACGACACAACA (SEQ ID NO. 9) |
| | Forward: GCGCGTGGAAGACTAGTGATTT (SEQ ID |
| | NO. 10) |
| | Reverse: AGTGCAGGGTCCGAGGTATT (SEQ ID |
| | NO. 11) |
| Has-miR- | Reverse transcription: |
| 98-5p | GTCGTATCCAGTGCAGGGTCCGAGGTATTCGCACTGGAT |
| | ACGACAACAAT (SEQ ID NO. 12) |
| | Forward: CGCGCGTGAGGTAGTAAGTTGT (SEQ ID |
| | NO. 13) |
| | Reverse: AGTGCAGGGTCCGAGGTATT (SEQ ID |
| | NO. 14) |
| Has-miR- | Reverse transcription: |
| 193a-5p | GTCGTATCCAGTGCAGGGTCCGAGGTATTCGCACTGGAT |
| | ACGACTCATCT (SEQ ID NO. 15) |
| | Forward: TGGGTCTTTGCGGGCG (SEQ ID NO. |
| | 16) |
| | Reverse: AGTGCAGGGTCCGAGGTATT (SEQ ID |
| | NO. 17) |
| Has-miR- | Reverse transcription: |
| 345-5p | GTCGTATCCAGTGCAGGGTCCGAGGTATTCGCACTGGAT |
| | ACGACGAGCCC (SEQ ID NO. 17) |
| | Forward: GCGGCTGACTCCTAGTCCA (SEQ ID |
| | NO. 19) |
| | Reverse: AGTGCAGGGTCCGAGGTATT (SEQ ID |
| | NO. 20) |
| Has-miR- | Reverse transcription: |
| 378a-3p | GTCGTATCCAGTGCAGGGTCCGAGGTATTCGCACTGGAT |
| | ACGACGCCTTC (SEQ ID NO. 21) |
| | Forward: CGCGACTGGACTTGGAGTCA (SEQ ID |
| | NO. 22) |
| | Reverse: AGTGCAGGGTCCGAGGTATT (SEQ ID |
| | NO. 23) |

The present invention also provides application of a miR-7-5p mimic in preparation of a small-nucleic-acid drug for inhibiting migration and invasion of breast cancer.

The present invention further demonstrates that the miR-7-5p mimic can significantly inhibit migration and invasion of breast cancer in vitro by conducting a wound healing assay and a Transwell migration and invasion assay using the miR-7-5p mimic. The present invention still further demonstrates that the miR-7-5p mimic can inhibit migration and invasion of breast cancer in vitro by targeted inhibition of RYK gene expression.

In the screening process of the present invention, the miRNAs screened out include miR-7-5p, miR-98-5p, miR-193a-5p, miR-345-5p, and miR-378a-3p.

Beneficial effects of the present invention are as follows: the present invention is directed to provide application of a miR-7-5p mimic in significantly inhibiting invasion and metastasis of breast cancer, and it has been found that the miR-7-5p mimic generates a significant inhibitory effect on breast cancer through targeted inhibition of the molecular mechanism of RYK. In vitro culture system, the miR-7-5p mimic can function to inhibit migration and invasion capabilities of breast cancer. The miR-7-5p mimic can inhibit RYK protein and mRNA levels in breast cancer. In nude mice, the miR-7-5p mimic can also significantly inhibit migration and invasion capabilities of breast cancer. Therefore, the present invention demonstrates that the miR-7-5p mimic can be a small-nucleic-acid drug to significantly inhibit breast cancer metastasis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a volcano plot showing differentially expressing miRNAs in exosomes derived from MDA-MB-231 and MCF7 through bioinformatics analysis.

FIG. 2 includes graphs showing identification of exosomes.

FIG. 3 includes graphs showing expression of miRNAs in cells and exosomes.

A, B and C of FIG. 4 includes graphs showing the effect of the miR-7-5p mimic on inhibition of migration and invasion of breast cancer.

A, B, C, D and E of FIG. 5 includes graphs showing the result of the effect of the miR-7-5p mimic on targeted inhibition of the RYK gene.

### DESCRIPTION OF THE EMBODIMENTS

The present invention will be further described in detail through specific embodiments, but it is not construed as limiting the scope of the present invention to the following examples. Without departing from the methods and ideas above of the present invention, various substitutions or modifications made according to the common technical knowledge and customary means in the field shall be included in the scope of the present invention. The assay methods without indicating specific conditions in the following embodiments are generally carried out according to conventional conditions or conditions suggested by manufacturers. Unless otherwise specified, materials, reagents, and the like used in the embodiment are those commercially available.

### Embodiment 1. Screening for Differentially Expressing miRNAs in Exosomes Derived from Differently Invasive Breast Cancer Cells Through Bioinformatics Analysis

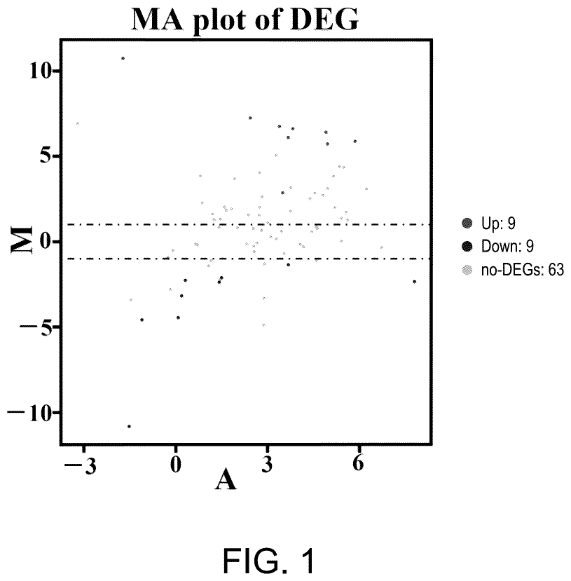

Original data was downloaded from two subsets, MDA231 EXO and MCF7 EXO, in dataset GSE114329 in GEO database, and was then filtered and processed to obtain clean sequences, which were then used to screen for differentially expressing genes. There were nine miRNAs screened out that had high expression in exosomes derived from a low-invasive breast cancer cell line, and the results are shown in FIG. 1. Several miRNAs with large differences in duplicate data were further excluded, and finally 5 miRNAs were selected, including miR-7-5p, miR-98-5p, miR-193a-5p, miR-345-5p, and miR-378a-3p.

### Embodiment 2. Validation of Differentially Expressing miRNAs in Exosomes Derived from Differently Invasive Breast Cancer Cells Screened Out

(1) Cell Culture

MDA-MB-231 and MCF7 cell lines were purchased from Dalian Meilun Biological Co., Ltd. Both cell lines were cultured in high-sugar DMEM media (Solarbio) containing 10% fetal bovine serum (BI) and in a cell culture incubator at 5% CO 2 and 37° C. In order to obtain exosomes unaffected by fetal bovine serum exosomes, the cells were also cultured in high-sugar DMEM media containing 10% exosome-free fetal bovine serum (SBI). The cells were cultured to logarithmic growth stage for assays.

(2) Exosome Extraction and Identification

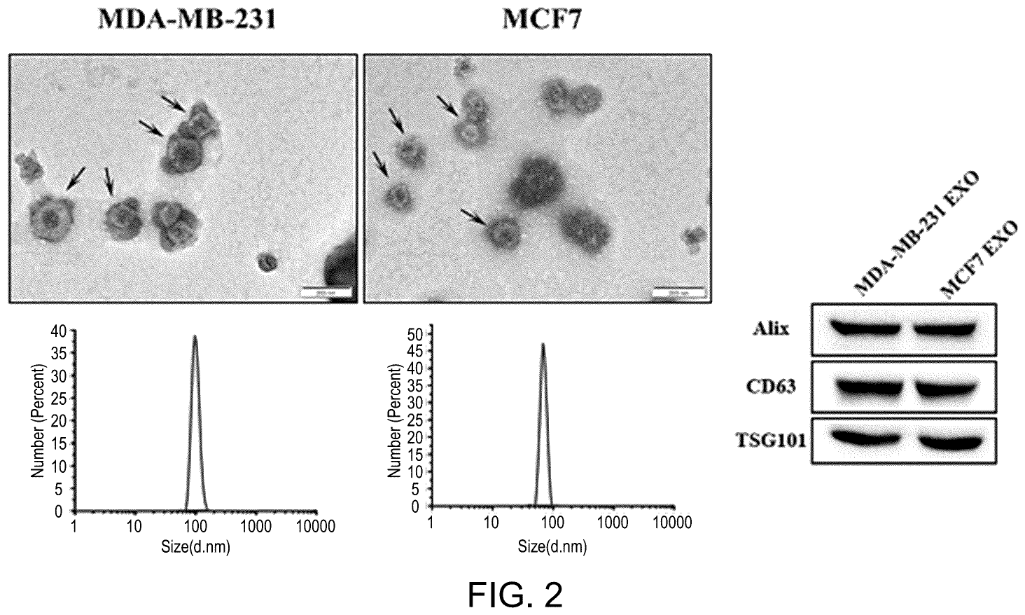

The cell culture supernatant was collected during the cell culture process, and the cell culture supernatant was subjected to high-speed centrifugation: 300×g centrifugation at 4° C. for 10 min, 2000×g centrifugation at 4° C. for 15 min, and 10000×g centrifugation at 4° C. for 30 min, followed by ultracentrifugation: the supernatant was subjected to 110000×g centrifugation at 4° C. for 90 min, then the supernatant was discarded, and the precipitate was resuspended in pre-cooled PBS and further subjected to 110000×g centrifugation at 4° C. for 90 min, and the precipitate was resuspended in proper PBS. The morphology was observed by transmission electron microscopy. The particle size was measured using a Zeta potential and nano-particle size analyzer. Exosome marker proteins CD63, TSG101, and Alix were detected using a Western Blot assay. The results are shown in FIG. 2. The results show that the exosomes were extracted.

(3) qRT-PCR Assay

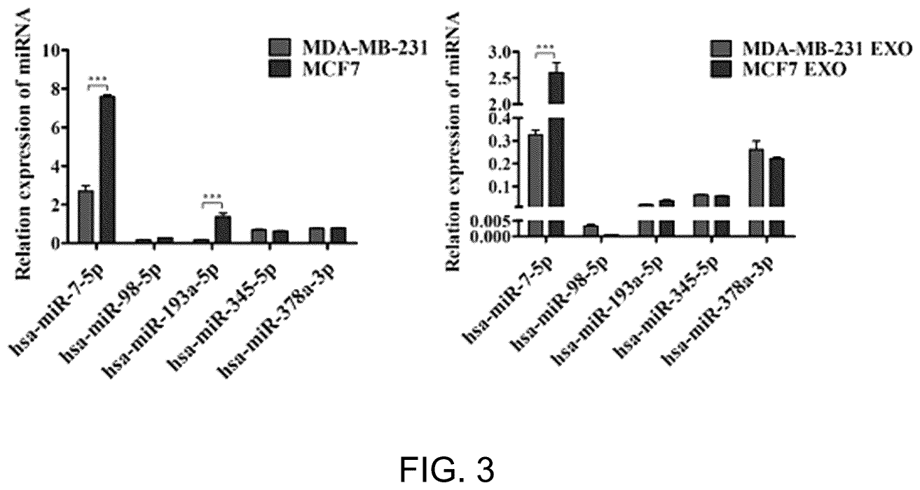

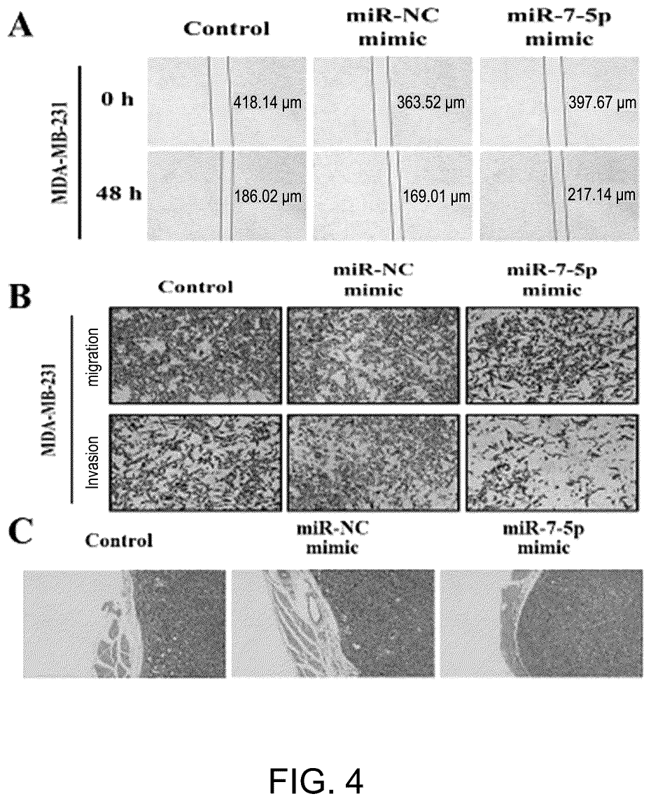

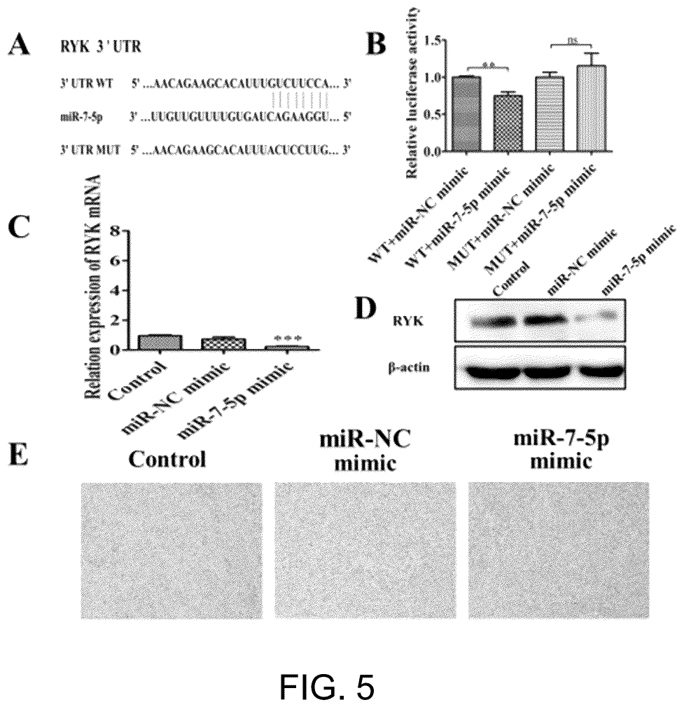

The miRNAs were extracted from cells and exosomes with RNAiso (Takara Bio) specialized in small RNA extraction. The miRNAs were reversely transcribed using miRNA 1st Strand cDNA Synthesis Kit (Vazyme). Real-time quantitative analysis of RNA levels was performed using SYBR Green reagent (Vazyme). Real-time analysis was performed according to the following protocol: pre-denaturation at 95° C. for 5 min, and 40 cycles including 95° C. for 15 sec, 60° C. for 20 sec, and 72° C. for 40 sec. The internal reference was U6. The results are shown in FIG. 3. Primer sequences of the miRNAs are shown in Table 1. The results show that miR-7-5p is the miRNA with the most significant differential expression, so it is hypothesized that the miR-7-5p can be a small-nucleic-acid drug to significantly inhibit breast cancer metastasis.

TABLE 1

| Genes | Primer sequences (5′-3′) |
|---|---|
| RYK | Forward: CTTTATCAGTGTTTCGGGTAG (SEQ ID |
| | NO. 2) |
| | Reverse: GCGTAGAAGTGGTTGGAGC (SEQ ID |
| | NO. 3) |
| GADPH | Forward: AGCCACATCGCTCAGACAC (SEQ ID |
| | NO. 4) |
| | Reverse: GCCCAATACGACCAAATCC (SEQ ID |
| | NO. 5) |
| U6 | Reverse transcription: |
| | TGGTGTCGTGGAGTCG (SEQ ID NO. 6) |
| | Forward: CTCGCTTCGGCAGCACA (SEQ ID |
| | NO. 7) |
| | Reverse: AACGCTTCACGAATTTGCGT (SEQ ID |
| | NO. 8) |
| Has-miR- | Reverse transcription: |
| 7-5p | GTCGTATCCAGTGCAGGGTCCGAGGTATTCGCACTGGAT |
| | ACGACACAACA (SEQ ID NO. 9) |
| | Forward: GCGCGTGGAAGACTAGTGATTT (SEQ ID |
| | NO. 10) |
| | Reverse: AGTGCAGGGTCCGAGGTATT (SEQ ID |
| | NO. 11) |
| Has-miR- | Reverse transcription: |
| 98-5p | GTCGTATCCAGTGCAGGGTCCGAGGTATTCGCACTGGAT |
| | ACGACAACAAT (SEQ ID NO. 12) |
| | Forward: CGCGCGTGAGGTAGTAAGTTGT (SEQ ID |
| | NO. 13) |
| | Reverse: AGTGCAGGGTCCGAGGTATT (SEQ ID |
| | NO. 14) |
| Has-miR- | Reverse transcription: |
| 193a-5p | GTCGTATCCAGTGCAGGGTCCGAGGTATTCGCACTGGAT |
| | ACGACTCATCT (SEQ ID NO. 15) |
| | Forward: TGGGTCTTTGCGGGCG (SEQ ID NO. |
| | 16) |
| | Reverse: AGTGCAGGGTCCGAGGTATT (SEQ ID |
| | NO. 17) |
| Has-miR- | Reverse transcription: |
| 345-5p | GTCGTATCCAGTGCAGGGTCCGAGGTATTCGCACTGGAT |
| | ACGACGAGCCC (SEQ ID NO. 17) |
| | Forward: GCGGCTGACTCCTAGTCCA (SEQ ID |
| | NO. 19) |
| | Reverse: AGTGCAGGGTCCGAGGTATT (SEQ ID |
| | NO. 20) |
| Has-miR- | Reverse transcription: |
| 378a-3p | GTCGTATCCAGTGCAGGGTCCGAGGTATTCGCACTGGAT |
| | ACGACGCCTTC (SEQ ID NO. 21 |
| | Forward: CGCGACTGGACTTGGAGTCA (SEQ ID |
| | NO. 22) |
| | Reverse: AGTGCAGGGTCCGAGGTATT (SEQ ID |
| | NO. 23) |

### Embodiment 3. miR-7-5p Mimic can Significantly Inhibit Migration and Invasion of Breast Cancer

(1) Cell Transfection Assay

Cells were cultured to about 80% confluence. Diluted LipoRNAiMAX reagent (Mei5bio), miR-7-5p mimic, and a negative control were used as directed and added to the cell culture media and incubated for more than 24 hours. The sequences of the miRNA mimic and the negative control are shown in Table 2.

TABLE 2

| Oligonucleotides | Sequences (5′-3′) |
|---|---|
| Mimics negative | Sense: UUCUCCGAACGUGUCACGUTT |
| control | (SEQ ID NO. 24) |
| | Antisense: ACGUGACACGUUCGGAGAATT |
| | (SEQ ID NO. 25) |
| Has-miR-7-5p | Sense: UGGAAGACUAGUGAUUUUGUUGUU |
| mimics | (SEQ ID NO. 26) |
| | Antisense: CAACAAAAUCACUAGUCUUCC |
| | AUU (SEQ ID NO. 27) |

(2) Wound Healing Assay

The treated cells were inoculated on 6-well plates and cultured to about 80% confluence, and then 3 layers of cells were scratched with a 10 μL tip at an equal distance in each of the wells. The cells were then washed twice with PBS and new cell culture medium was added, and then scratch widths were measured with an inverted microscope. A pre-treated and liposome-encapsulated miR-7-5p mimic or negative control was added and an additional 48 hours was allowed for incubation before measuring the scratch widths again. Cell migration was evaluated through the wound healing rate, and the wound healing rate=(0 h scratch width−24 h scratch width)/0 h scratch width×100%. The results are shown in A of FIG. 4. The results also indicate that miR-7-5p mimic can significantly inhibit migration of breast cancer cells.

(3) Transwell Migration and Invasion Assay

The migration and invasion capabilities of cells were detected using polycarbonate membrane Transwell chambers with a bottom pore size of 8 μm. Transfected cells were resuspended in serum-free DMEM medium and added to the upper chamber (coated with matrigel or uncoated), and 750 μL of DMEM complete medium containing 10% serum was added to the lower chamber. Incubation was allowed in an incubator for 24 hours. The cells were first fixed with 4% paraformaldehyde for 2 min and then permeabilized in methanol for 20 min. Finally, the cells were stained with 0.1% crystal violet solution for 15 min. The cells that did not migrate in the upper chamber layer, as well as the matrigel coating were then gently scraped off with a cotton swab, and a microscope was used for observation. The results are shown in B of FIG. 4. The results show that miR-7-5p mimic can significantly inhibit migration and invasion of breast cancer cells in vitro.

(4) Establishment of Subcutaneous Tumor Model in Tumor-Bearing Nude Mice

4-week-old female BALB/c nude mice weighing about 12-16 g were selected. The nude mice were acclimatized for one week after arrival, and food, water, and bedding were strictly sterilized and prepared adequately. The resuscitated MDA-MB-231 cells were subcultured for 3 times. After centrifugation of the cells, a cell suspension (2.5×10⁷ cells/mL) was prepared with serum-free DMEM medium, and 100 μL of the cell suspension was subcutaneously injected at the axilla of the nude mice to construct a subcutaneous tumor model of breast cancer in nude mice. When the tumor volume of the tumor-bearing nude mice increased to about 100 mm 3, the nude mice were randomly divided into 3 groups (n=3) and injected with the saline, miR-7-5p mimic, and negative control, separately, at multiple sites within the tumors once every two days for 7 times.

(6) H & E Staining

Tissues of tumors was taken from the nude mice. The tumors were first fixed with 4% paraformaldehyde and then conventionally embedded with paraffin and sectioned. They were then deparaffinized in xylene and varying concentrations of ethanol. Afterwards, they were washed with distilled water for 2 min. Next, they were stained with hematoxylin and eosin, and it is noted that the excess staining solution was rinsed off after each staining. After that, they were conventionally dehydrated and hyalinized with ethanol and xylene again, and then mounted with neutral resin. They were observed with a microscope after drying. The results are shown in C of FIG. 4. The results show that the tissue of the group of tumors which were injected with the miR-7-5p mimic had an edge that was more distinct, which indicated that miR-7-5p could also significantly inhibit migration and invasion of breast cancer cells in vivo.

### Embodiment 4. miR-7-5p Mimic can Inhibit Migration and Invasion of Breast Cancer by Targeted Inhibition of RYK Gene Expression

(1) Dual Luciferase Reporter Gene Assay

The pmirGLO vectors for RYK-WT and RYK-MUT were constructed by Shanghai GenePharma (A of FIG. 5). MDA-MB-231 cells at logarithmic growth stage were inoculated on 12-well plates. A mimic negative control or miR-7-5p mimic was co-transfected with RYK-WT and RYK-MUT into MDA-MB-231 cells when the cells were cultured to about 70% confluence. After 48 hours of transfection, the cells were lysed, and luciferase activity was detected by a luciferase assay kit. The results are shown in B of FIG. 5. The results show that miR-7-5p mimic is capable of targeted inhibition of RYK gene.

(2) Western Blot Assay

Cells or exosomes were first lysed with lysis buffer and then incubated on ice for 15 min, and then the supernatant was collected after centrifugation. Protein concentrations were determined by a BCA protein assay kit. The protein samples were separated with 10% SDS-polyacrylamide gel, and then the protein bands were transferred onto 0.45 μm PVDF membranes, which were then sealed with 5% non-fat dry milk. The sealed membranes and corresponding primary antibody dilutions were incubated at 4° C. overnight and then washed with TBST buffer for four times. After that, the membranes and secondary antibody dilutions were incubated at 4° C. for 4-5 hours. After secondary antibody incubation, the membranes were further washed with TBST buffer, and the protein bands on the membranes were observed using ECL luminescent liquid and an imaging system. The results are shown in D of FIG. 5. The results show that the protein expression of RYK gene in MDA-MB-231 cells was significantly decreased after transfection of the mimic, thus demonstrating that the miR-7-5p mimic is capable of targeted inhibition of RYK gene expression.

(3) qRT-PCR Assay

The mRNAs were extracted from cells and exosomes with RNAiso (Takara Bio). The mRNAs were reversely transcribed using HiScript II 1st Strand cDNA Synthesis Kit (Vazyme). Real-time quantitative analysis of RNA levels was performed using SYBR Green reagent (Vazyme). Real-time analysis was performed according to the following protocol: pre-denaturation at 95° C. for 5 min, and 40 cycles including 95° C. for 10 sec and 60° C. for 30 sec. The internal reference was GADPH. The results are shown in A, B, C, D and E of FIG. 5. Primer sequences of the RYK mRNAs are shown in Table 1. The results show (C of FIG. 5) that miR-7-5p can reduce expression of RYK mRNAs as well, demonstrating that miR-7-5p mimic is capable of targeted inhibition of RYK gene expression.

(4) Immunohistochemistry

Paraffin embedding, sectioning, and deparaffinization were performed in the same manner as in H & E staining. The tumors had endogenous catalase removed and were then washed. After adding citrate buffer, the tumors were placed in a microwave oven for antigen retrieval. Relevant antibodies were then incubated with serum sealing. SABC was then added for incubation for half an hour, and a color developer was added after washing. Hematoxylin was then used for counterstaining, followed by ethanol and xylene for dehydration. Finally, the tumors were mounted with neutral resin, and observed with a microscope after drying. The results show (E of FIG. 5) that miR-7-5p can reduce RYK expression as well, demonstrating that miR-7-5p mimic is also capable of targeted inhibition of RYK gene expression in vivo.

What is claimed is:

1. A miR-7-5p mimic for inhibiting migration and invasion of breast cancer, wherein a sequence of miR-7-5p is shown in SEQ ID NO.1: UGGAAGACUAGUGAUUUUGUUGUU.

2. The miR-7-5p mimic according to claim 1, wherein a sequence of the miR-7-5p mimic is as follows:

| | Guide strand: |
|---|---|
| (SEQ ID NO. 1) | |
| | UGGAAGACUAGUGAUUUUGUUGUU; |
| | Passenger strand: |
| (SEQ ID NO. 27) | |
| | CAACAAAAUCACUAGUCUUCCAUU. |

3. A screening method for the miR-7-5p mimic according to claim 1, comprising following steps:
step (1) utilizing two subsets, MDA231 EXO and MCF7 EXO, in dataset GSE114329 under GEO database for bioinformatics analysis, downloading original data from the dataset GSE114329 and filtering and processing the original data, and then screening for differentially expressing genes; and
step (2) subjecting screened differentially expressing miRNAs to qRT-PCR analysis to screen out the miR-7-5p mimic.

4. The screening method according to claim 3, wherein in the step (2), a procedure of the qRT-PCR analysis comprises: pre-denaturation at 95° C. for 5 min, and 40 cycles including 95° C. for 15 sec, 60° C. for 20 sec, and 72° C. for 40 sec, with an internal reference being U6.

5. The screening method according to claim 3, wherein in the qRT-PCR analysis, primer sequences of the miRNAs are specifically as follows:

| Genes | Primer sequences (5′-3′) |
|---|---|
| RYK | Forward: CTTTATCAGTGTTTCGGGTAG (SEQ ID |
| | NO. 2) |
| | Reverse: GCGTAGAAGTGGTTGGAGC (SEQ ID |
| | NO. 3) |
| GADPH | Forward: AGCCACATCGCTCAGACAC (SEQ ID |
| | NO. 4) |
| | Reverse: GCCCAATACGACCAAATCC (SEQ ID |
| | NO. 5) |
| U6 | Reverse transcription: |
| | TGGTGTCGTGGAGTCG (SEQ ID NO. 6) |
| | Forward: CTCGCTTCGGCAGCACA (SEQ ID |
| | NO. 7) |
| | Reverse: AACGCTTCACGAATTTGCGT (SEQ ID |
| | NO. 8) |
| Has-miR- | Reverse transcription: |
| 7-5p | GTCGTATCCAGTGCAGGGTCCGAGGTATTCGCACTGGAT |
| | ACGACACAACA (SEQ ID NO. 9) |
| | Forward: GCGCGTGGAAGACTAGTGATTT (SEQ ID |
| | NO. 10) |
| | Reverse: AGTGCAGGGTCCGAGGTATT (SEQ ID |
| | NO. 11) |
| Has-miR- | Reverse transcription: |
| 98-5p | GTCGTATCCAGTGCAGGGTCCGAGGTATTCGCACTGGAT |
| | ACGACAACAAT (SEQ ID NO. 12) |
| | Forward: CGCGCGTGAGGTAGTAAGTTGT (SEQ ID |
| | NO. 13) |
| | Reverse: AGTGCAGGGTCCGAGGTATT (SEQ ID |
| | NO. 14) |
| Has-miR- | Reverse transcription: |
| 193a-5p | GTCGTATCCAGTGCAGGGTCCGAGGTATTCGCACTGGAT |
| | ACGACTCATCT (SEQ ID NO. 15) |
| | Forward: TGGGTCTTTGCGGGCG (SEQ ID NO. |
| | 16) |
| | Reverse: AGTGCAGGGTCCGAGGTATT (SEQ ID |
| | NO. 17) |
| Has-miR- | Reverse transcription: |
| 345-5p | GTCGTATCCAGTGCAGGGTCCGAGGTATTCGCACTGGAT |
| | ACGACGAGCCC (SEQ ID NO. 17) |
| | Forward: GCGGCTGACTCCTAGTCCA (SEQ ID |
| | NO. 19) |
| | Reverse: AGTGCAGGGTCCGAGGTATT (SEQ ID |
| | NO. 20) |
| Has-miR- | Reverse transcription: |
| 378a-3p | GTCGTATCCAGTGCAGGGTCCGAGGTATTCGCACTGGAT |
| | ACGACGCCTTC (SEQ ID NO. 21) |
| | Forward: CGCGACTGGACTTGGAGTCA (SEQ ID |
| | NO. 22) |
| | Reverse: AGTGCAGGGTCCGAGGTATT (SEQ ID |
| | NO. 23) |

6. An application of the miR-7-5p mimic according to claim 1 in preparation of a small-nucleic-acid drug for inhibiting migration and invasion of breast cancer.


SEQUENCE LISTING (27 sequences)

SEQ ID NO: 1 - protein, 24 aa, synthetic construct
UGGAAGACUAGUGAUUUUGUUGUU

SEQ ID NO: 2 - DNA, 21 bp, synthetic construct
  source 1..21: mol_type = other DNA
ctttatcagtgtttcgggtag

SEQ ID NO: 3 - DNA, 19 bp, synthetic construct
  source 1..19: mol_type = other DNA
gcgtagaagtggttggagc

SEQ ID NO: 4 - DNA, 19 bp, synthetic construct
  source 1..19: mol_type = other DNA
agccacatcgctcagacac

SEQ ID NO: 5 - DNA, 19 bp, synthetic construct
  source 1..19: mol_type = other DNA
gcccaatacgaccaaatcc

SEQ ID NO: 6 - DNA, 16 bp, synthetic construct
  source 1..16: mol_type = other DNA
tggtgtcgtggagtcg

SEQ ID NO: 7 - DNA, 17 bp, synthetic construct
  source 1..17: mol_type = other DNA
ctcgcttcggcagcaca

SEQ ID NO: 8 - DNA, 20 bp, synthetic construct
  source 1..20: mol_type = other DNA
aacgcttcacgaatttgcgt

SEQ ID NO: 9 - DNA, 50 bp, synthetic construct
  source 1..50: mol_type = other DNA
gtcgtatccagtgcagggtccgaggtattcgcactggatacgacacaaca

SEQ ID NO: 10 - DNA, 22 bp, synthetic construct
  source 1..22: mol_type = other DNA
gcgcgtggaagactagtgattt

SEQ ID NO: 11 - DNA, 20 bp, synthetic construct
  source 1..20: mol_type = other DNA
agtgcagggtccgaggtatt

SEQ ID NO: 12 - DNA, 50 bp, synthetic construct
  source 1..50: mol_type = other DNA
gtcgtatccagtgcagggtccgaggtattcgcactggatacgacaacaat

SEQ ID NO: 13 - DNA, 22 bp, synthetic construct
  source 1..22: mol_type = other DNA
cgcgcgtgaggtagtaagttgt

SEQ ID NO: 14 - DNA, 20 bp, synthetic construct
  source 1..20: mol_type = other DNA
agtgcagggtccgaggtatt

SEQ ID NO: 15 - DNA, 50 bp, synthetic construct
  source 1..50: mol_type = other DNA
gtcgtatccagtgcagggtccgaggtattcgcactggatacgactcatct

SEQ ID NO: 16 - DNA, 16 bp, synthetic construct
  source 1..16: mol_type = other DNA
tgggtctttgcgggcg

SEQ ID NO: 17 - DNA, 20 bp, synthetic construct
  source 1..20: mol_type = other DNA
agtgcagggtccgaggtatt

SEQ ID NO: 18 - DNA, 50 bp, synthetic construct
  source 1..50: mol_type = other DNA
gtcgtatccagtgcagggtccgaggtattcgcactggatacgacgagccc

SEQ ID NO: 19 - DNA, 19 bp, synthetic construct
  source 1..19: mol_type = other DNA
gcggctgactcctagtcca

SEQ ID NO: 20 - DNA, 20 bp, synthetic construct
  source 1..20: mol_type = other DNA
agtgcagggtccgaggtatt

SEQ ID NO: 21 - DNA, 50 bp, synthetic construct
  source 1..50: mol_type = other DNA
gtcgtatccagtgcagggtccgaggtattcgcactggatacgacgccttc

SEQ ID NO: 22 - DNA, 20 bp, synthetic construct
  source 1..20: mol_type = other DNA
cgcgactggacttggagtca

SEQ ID NO: 23 - DNA, 20 bp, synthetic construct
  source 1..20: mol_type = other DNA
agtgcagggtccgaggtatt

SEQ ID NO: 24 - protein, 21 aa, synthetic construct
UUCUCCGAACGUGUCACGUTT

SEQ ID NO: 25 - protein, 21 aa, synthetic construct
ACGUGACACGUUCGGAGAATT

SEQ ID NO: 26 - protein, 24 aa, synthetic construct
UGGAAGACUAGUGAUUUUGUUGUU

SEQ ID NO: 27 - protein, 24 aa, synthetic construct
CAACAAAAUCACUAGUCUUCCAUU